(19) United States
(12) Patent Application Publication
(10) Pub. No.: US 2006/0122460 A1
(43) Pub. Date: Jun. 8, 2006

(54) Airway management

(76) Inventor: Henry Kamali, Los Gatos, CA (US)

Correspondence Address: KOKKA & HSU, PC, 1001 N. RENGSTROFF AVE., SUITE 520, MOUNTAIN VIEW, CA, 94043-1748, US

(21) Appl. No.: 11/007,385

(22) Filed: Dec. 7, 2004

(51) Int. Cl. A61B 1/04

(52) U.S. Cl. 600/120; 600/109

(57) ABSTRACT

Airway management is described, including an imaging device configured to capture an image of the airway and convert the image into a signal, a stylet configured to guide placement of an endotracheal tube in the airway and conduct the signal from the imaging device, the stylet being configured for introduction into a lumen of the endotracheal tube, the stylet and endotracheal tube being inserted into the airway, and an image processor configured to receive and process the signal from the imaging device, the image being sent to a display for use in guiding placement of the endotracheal tube. Also described are techniques for airway management, including capturing an image of the airway, converting the image into a signal, conducting the signal using an electrically conductive and malleable stylet, processing the signal to display the image of the airway, and guiding the insertion of an endotracheal tube into the airway using an airway management system, the airway management system having an imaging device configured to capture the image and a display for viewing the image.

### FIELD OF THE INVENTION

The present invention relates generally to anesthesiology. Specifically, airway management is described.

### BACKGROUND OF THE INVENTION

When a patient undergoes a surgical procedure that requires general anesthesia, medication is administered and unconsciousness occurs. Immediately following unconsciousness, the patient becomes apneic (i.e., stops breathing). A person qualified in airway management (e.g., anesthesiologist, nurse anesthetist, or other medical personnel) has a brief period of time in which to secure an airway or provide an adequate means of artificial ventilation and oxygenation. Some conventional solutions used to secure an airway include intubation using an endotracheal tube (ETT). Intubation involves placing an ETT in a patient's airway, often providing an outer seal between the ETT and the trachea to prevent air from passing around instead of through the ETT. In some conventional solutions, an ETT may have an inflatable balloon that may be inflated to create a seal between the tracheal passage and the external surface of an ETT.

After unconsciousness is achieved in the supine position, a laryngoscope may be placed in the mouth of the patient to gain a view of the patient's vocal cords to aid placement of an ETT. The vocal cords are anatomically located at the opening of the trachea (i.e., windpipe), which leads to the lungs and bronchial structures and passages. An ETT is then slipped between the vocal cords and into the trachea and the laryngoscope is removed. The ETT is then connected to an oxygen source and mechanical ventilation is initiated.

However, there are various problems associated with conventional airway management techniques. For example, conventional airway management equipment (e.g., laryngoscope) may be either bulky or unsuitable for a variety of anatomical factors or physiologic conditions, thus reducing the likelihood of successful of intubation. Factors such as a small mouth opening, large teeth and tongue size, poor neck mobility, inadequate mandibular space (i.e., thyromental distance), small chin, arched palate, short neck, prominent Adam's apple, or poor patient positioning are factors that may inhibit airway management. Moreover, common disorders such as arthritis, diabetes, trauma, infections, Down's syndrome, and obesity may also cause problems leading to misplacement of an ETT or difficulty airway management. There can also be considerable damage rendered to the vocal cords or other oropharyngeal structures as attempts are made to secure the airway. Conventional solutions may also lead to limited or no visibility of the vocal cords as a laryngoscope is inserted and may result in inability to correctly place an ETT. Specifically, the ETT may be placed into the esophagus instead of the trachea.

This may be particularly problematic if the patient has stopped breathing or there is a limited amount of time in which to initiate ventilation and oxygenation and can result in patient morbidity or mortality. Conversely, even with intubation of the trachea, the ETT may be inserted too far causing misplacement into the right or left main stem bronchus thereby leading to inadequate oxygenation.

Thus, what is needed is a solution for managing an airway overcoming the limitations of conventional techniques.

### BRIEF DESCRIPTION OF THE DRAWINGS

Various embodiments of the invention are disclosed in the following detailed description and the accompanying drawings:

FIG. 1 illustrates an exemplary endotracheal tube;

FIG. 2A illustrates an exemplary stylet imaging system;

FIG. 2B illustrates an exemplary airway management system;

FIG. 3 illustrates an alternative exemplary airway management system including a local display;

FIG. 4 illustrates an exemplary display connector assembly for an airway management system having a local display.;

FIG. 5A illustrates an exemplary cross-sectional diagram of a stylet imaging system;

FIG. 5B illustrates an alternative exemplary cross-sectional diagram of a stylet imaging system;

FIG. 6 illustrates an exemplary transverse diagram of a distal end of an airway management system;

FIG. 7A is a block diagram illustrating an exemplary airway management system;

FIG. 7B is a block diagram illustrating an alternative exemplary airway management system;

FIG. 7C is a block diagram illustrating yet another alternative exemplary airway management system; and

FIG. 8 is a block diagram illustrating an exemplary computer system suitable for implementing airway management.

### DETAILED DESCRIPTION OF THE PREFERRED EMBODIMENTS

Implementation of described techniques may occur in numerous ways, including as a system, device, apparatus, process, a computer readable medium such as a computer readable storage medium, or a computer network wherein program instructions are sent over optical or electronic communication links.

A detailed description of one or more embodiments is provided below along with accompanying figures that illustrate the principles of the embodiments. The scope of the embodiments is limited only by the claims and encompasses numerous alternatives, modifications and equivalents. Numerous specific details are set forth in the following description. These details are provided solely for the purposes of example and the embodiments may be practiced according to the claims without some or all of these specific details.

Airway management techniques using an ETT and stylet camera are described. By using an airway management system, an ETT may be placed within an airway safely and accurately, avoiding damage to a patient's oropharyngeal structures and enabling the management of difficult airways. A stylet imaging system, including an imaging device, image processor and display, may be introduced into an ETT, creating an airway management system. An airway management system may then be inserted into an airway and, using an image provided by an imaging device, guide the placement of an ETT without damaging the vocal cords or other airway structures. Upon safe and accurate placement of the airway management system, the stylet imaging system may be retracted from the ETT, which is left in place within the airway. An airway management system enables accurate positioning and placement of an ETT for ventilation and oxygenation.

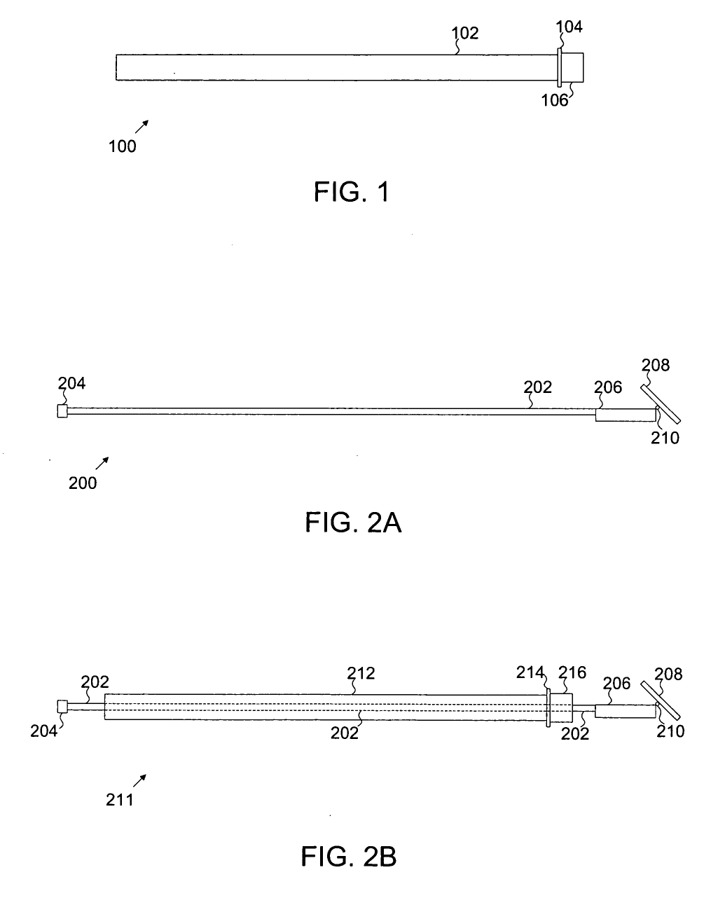

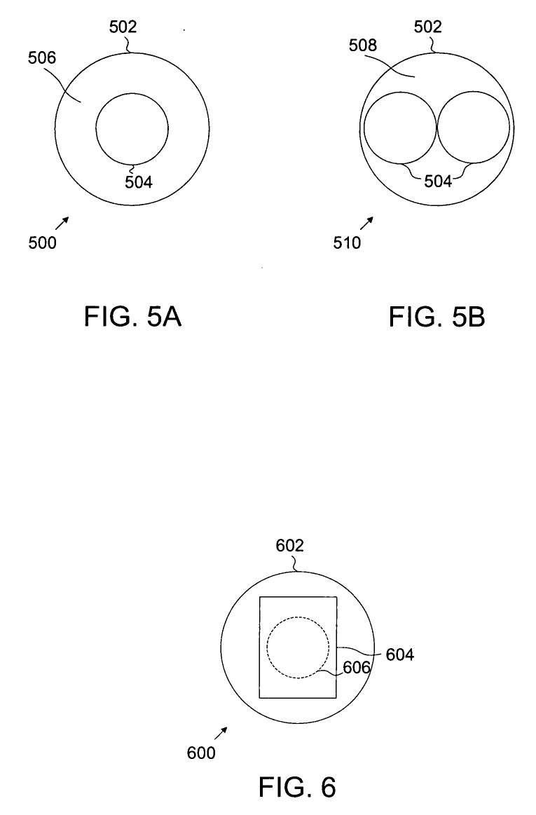

FIG. 1 illustrates an exemplary endotracheal tube system 100. Here, endotracheal tube system (“ETT system”) 100 includes an ETT 102, connector 104, and connector mouth 106. ETT 102 may be coupled to ventilation, oxygenation, or anesthesia-delivery equipment or accessories using connector 104 and connector mouth 106. In some examples, ETT 102 may be positioned in a patient's airway by bending or shaping ETT 102 to conform to the geometry of a patient's airway, regardless of physical, anatomical, or other factors that may affect positioning. In some examples, ETT 102 may be bent or constructed of flexible, malleable material (e.g., rubber, plastic, polyvinyl chloride (PVC), and the like), enabling ETT 102 to be deformed in order to fit a patient's airway. ETT 102 may include one or more lumens (not shown) configured to receive a stylet (not shown) or a stylet imaging system (described below in connection with FIG. 2A). One or more lumens may also be adapted to supply gas (e.g., oxygen, anesthesia, and the like) or provide suction to remove secretions. Lumens associated with ETT system 100 are described in greater detail below in connection with FIGS. 5A-5B. Here, ETT system 100 may be configured to receive a stylet imaging system, as described in greater detail in connection with FIG. 2A.

FIG. 2A illustrates an exemplary stylet imaging system 200. In this example, stylet imaging system 200 includes stylet 202, imaging device 204, image processor 206, display 208, and display connector 210. In some examples, stylet imaging system 200 may be inserted in an ETT (e.g., ETT system 100) and used to manage the placement of an ETT into an airway. Here, stylet 202 may be implemented using a flexible rod constructed from electrically conductive materials. Stylet 202 may be inserted within ETT system 100 or another airway management tube intended for insertion into a patient's airway. As an example, stylet 202 may be malleable or flexible. In other examples, stylet 202 may also be rigid or semi-rigid. Stylet 202 may also be bent or deformed into a variety of shapes. In some examples, stylet 202 may be placed within ETT system 100 and bent or twisted into a desired shape or configuration. In other examples, stylet 202 may also be disposable, with each stylet capable of being changed out between operations, patients, and the like. The deformable property of stylet 202 within an ETT system 100 enables placement of the ETT within an airway. Stylet 202 may also be metallic or composed of metallic alloys that exhibit electrically conductive properties such that electrical signals may be conducted between other components directly or indirectly coupled to stylet 202. Electrical signals may be transferred along stylet 202 at a voltage low enough to avoid significant loss in transmission and prevent heat-build up or damage either to ETT system 100 or tissue in the surrounding airway. In other examples, stylet 202 may be hollow or include a lumen for passing wires or filaments that may be used to also conduct electrical signals. In still other examples, a transmitter and receiver may be placed within stylet 202 for transmitting wireless signals to other components or devices in ETT system 100 or other external systems. For example, this may include transmitting (e.g., wired, wireless) signals from a camera or imaging device disposed at a distal end of stylet 202 to a display or processing unit remotely located.

As an example, stylet 202 may be made from materials (e.g., metals, metallic alloys, composite materials, and the like) that are malleable and also possess electrically conductive properties to enable the propagation of electromagnetic energy (e.g., RF waves or electrical signals) between imaging device 204 and display 208. In some examples, stylet 202 may be sheathed in a plastic, rubber, or other malleable, insulated coating to prevent the inadvertent loss of electrical signals or signal strength, as well as facilitate the introduction of stylet 202 into ETT system 100. Stylet 202 may be inserted into ETT system 100 and deformed for a given “fit” within a patient's airway. Imaging device 204 supplies an image in the form of electrical signals to display 208, which may be used to guide the placement of an ETT into an airway. Stylet imaging system 200, when used to intubate a patient, avoids potentially damaging a patient's vocal cords while enabling rapid and accurate placement of an ETT by providing a real-time image as placement occurs.

In some examples, stylet 202 may also be inserted or removed from ETT system 100 as a disposable, detachable component. In other examples, stylet 202 may be implemented with a light source at its distal tip. In yet other examples, stylet 202 may be of varying sizes and diameters to accommodate adult, pediatric, and multiple (e.g., double) lumen tubes. In some examples, display 208 may be implemented as a small, liquid crystal display (LCD) device coupled to stylet 202. In other examples, different types of displays may be implemented. Connector 210 may be used to locally couple display 208 to image processor 206.

In some examples, connector 210 may include electrical connectors (e.g., wires, metal contacts, and the like) that communicate signals between display 208 and image processor 206. In other examples, electrical connectors may be provided separately from connector 210. Connector 210 may also permit direct and indirect coupling of display 208 to image processor 206 as well as mechanical and electrical connections. As an example, connector 210 may be a “Y” swivel connection, as described below in connection with FIG. 4. In other examples, connector 210 may be implemented as a screw, clip, couple, slide, or other coupling assembly. In some examples, display 208 may also be remote from stylet imaging system 200, receiving signals sent using various wireless formats (e.g., RF waves, IEEE 802.11, Bluetooth, UHF, and the like).

Signals may be sent from image processor 206 or another component attached to stylet 202. In some examples, electrical signals may be communicated between imaging device 204 and image processor 206, using stylet 202. Once received at image processor 206, electrical signals may be processed and transmitted to a local or remote display for viewing. In other examples, signals may be converted to RF waves and radiated for reception at a remote receiver coupled to a remote display (not shown). As another example, a larger display (e.g., LCD, flat panel, endoscopy tower/cart/rack-mounted display, and the like) may also be used to enhance an image of a patient's airway as stylet imaging system 200 is placed.

Image processor 206 may be implemented using a variety of techniques. In some examples, a power supply (not shown) may be implemented externally to stylet imaging system 200. A power supply (not shown) such as a battery or external AC/DC converter may be coupled to image processor 206. Power supplies may be implemented as rechargeable, non-rechargeable, portable, or disposable batteries. In other examples, a battery (not shown) may be implemented as part of image processor 206, supplying power to stylet imaging system 200 and its associated components, including imaging device 204. In still other examples, a power source may be implemented as another attachment to stylet imaging system 200. Alternatively, a light (not shown) may be included with or coupled to imaging device 204 and power may be supplied from either an internal (i.e., a battery within image processor 206) or external power supply. In some examples, stylet imaging system 200 may be portable. In other examples, stylet imaging system 200 may be disposable and, in some examples, image processor 206 may be detached from stylet imaging system 200 and coupled to a replacement stylet imaging system. In still other examples, stylet imaging system 200 may include a wireless transceiver (not shown) for sending and receiving signals (e.g., RF) from image processor 206 to a remote device or system (e.g., display, endoscopy tower, supplemental display device, computer, server, video recorder, and the like).

FIG. 2B illustrates an exemplary system for airway management. Here, an overall system 211 is shown, including stylet 202, imaging device 204, image processor 206, display 208, display connector 210, ETT 212, connector 214, and connector mouth 216. Here, stylet imaging system 200 (FIG. 2A) may be introduced (i.e., inserted) into connector mouth 216 to create airway management system 211. In this example, stylet 202 is shown partially extruding from the proximal and distal ends of ETT 212. In other examples, stylet 202 may be varied by length, either shorter or longer. Here, stylet 202 and imaging device 204 may have a smaller cross-sectional diameter than a lumen of ETT 212. This enables stylet imaging system 200 to be introduced and retracted (i.e., withdrawn) from ETT 212. In some examples, there is space between stylet imaging system 200 and ETT 212, which enables freedom of movement for introduction or retraction. While inserted, stylet imaging system 200 may be deformed and hold a particular shape, causing ETT 212 to be deformed in a similar shape. The deformation of airway management system 211 does not affect the electrically conductive properties of stylet imaging system 200. This exemplary configuration enables an image (e.g., real-time, still, delayed) of a patient's airway and vocal cords to be captured as ETT 212 is placed within the trachea. Imaging device 204 and stylet 202 are of such a diameter so as to allow retraction from ETT 212, which remains in place within a patient's airway, after safe and accurate placement. Generally, stylet imaging system 200 is introduced into an ETT and then the assembled system is placed into an airway after induction of general anesthesia or appropriate anesthetization. This may be performed with or without the aid of a laryngoscope using airway management system 211 (i.e., stylet imaging system 200 introduced into an ETT). In some examples, imaging device 204 and stylet 202 may have different diameters in order to accommodate ETTs of varying sizes for either adult or pediatric uses. In other examples, different devices or interchangeable components may be coupled to airway management system 200 or 211.

As an example, airway management system 200 or 211 may also be attached to a laryngotracheal anesthesia (LTA) kit. An LTA kit may be used to supply anesthesia using ETT 212 to a patient's airway prior to intubation or after intubation is complete. In some examples, other kits may be used to supply oxygen or other gases to a patient's airway as determined by an anesthesiologist. For example, atomized lidocaine (“lidofog”) may be injected into a patient's airway using airway management system 200 or 211, before an ETT has been introduced. Anesthesia may be used to anesthetize the oropharynx or associated respiratory structures. In some examples, ETT 202 or 212 may have a side port (not shown) that can be used as either a suction port to remove secretions or as an injection port to supply oxygen or anesthesia. Airway management system 211 may be used to intubate a patient either with or without performing laryngoscopy. Once ETT 212 has been introduced, stylet imaging system 200 may be retracted and ETT 212 may be secured and confirmed.

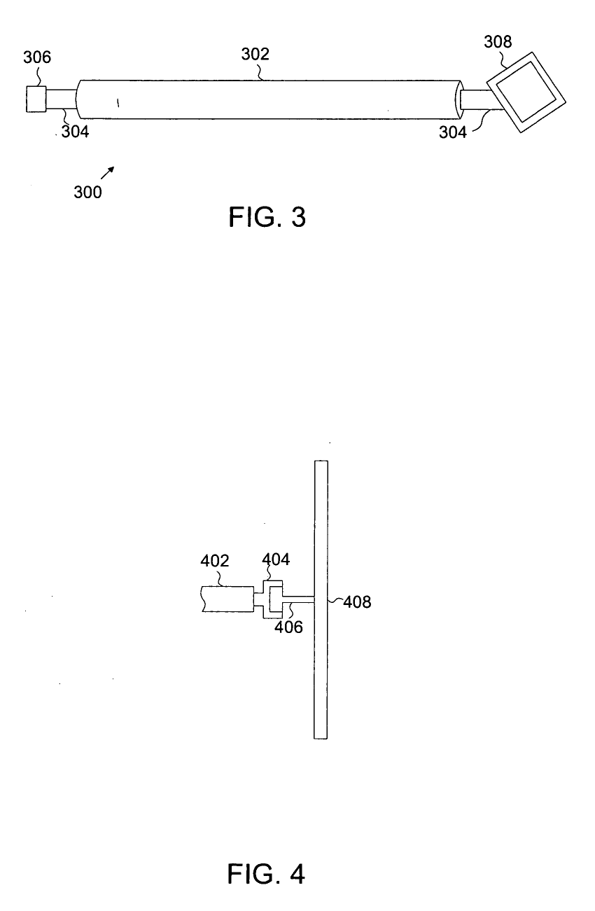

FIG. 3 illustrates an alternative exemplary system for airway management including a local display. Here, a side view of airway management system 300 is shown. In this example, airway management system 300 includes ETT 302, stylet 304, imaging system 306, and display 308. Display 308 may be implemented as described above and is shown tilted to a side to further permit viewing from a side angle. In some examples, display 308 may be manipulated, using a flexible coupling assembly (not shown) to enable an image of a patient's airway to be viewed at the screen of the display at various angles. In some examples, display 308 may be tiled or placed at different angles to permit an operator (e.g., anesthesiologist) to view an image on the screen while concurrently manipulating airway management system 300 for placement within a patient's airway. Upon completion of placing airway management system 300 within a patient's trachea, stylet 304 and imaging device 306 may be extracted from the patient's airway, leaving ETT 302 in place for securing and confirmation (confirmation refers to ensuring that ETT 302 has been properly placed before ventilation and oxygenation occurs).

FIG. 4 illustrates an exemplary display connector assembly for an airway management system having a local display. Here, a proximal end of stylet 402 is shown with female “Y” connector 404, male connector 406, and display 408. Display 408 may be implemented, in some examples, as a small (e.g., 1-2 inch) flat LCD screen or another display type, but is not limited to those displays shown or described. As an example, display 408 may also have an internal or external receiver coupled to it, providing the ability to receive electrical (e.g., RF) signals for transferring an image received at imaging device 204 or 306. In other examples, signals may be transferred wirelessly. Display 408 may be rigidly connected to the proximal end of male connector 406. In other examples, display 408 may be connected using a coupling having one or more degrees of freedom with male connector 406. As another example, female connector 404 and male connector 406 may be implemented using a single connector. Here, the coupling between female connector 404 and male connector 406 may also be “broken” by applying pressure to either twist or pull display 408 from stylet 402. This enables display 408 to be switched or replaced as well as replacing stylet 402. In some examples, stylet 402 may also be constructed of metal or metallic alloys that enable the conduction of electrical signals across female connector 404 and male connector 406 to display 408, eliminating the need for additional wires. This may enable a power supply (not shown) to provide power to or from display 408 as well as to other components coupled (i.e., directly or indirectly) to stylet 402 (e.g., imaging device 204, image processor 206, a light, and others). In still other examples, more or fewer intermediate connector components may be used in conjunction with female connector 404 and male connector 406.

FIG. 5A illustrates an exemplary cross-sectional diagram of an airway management system. Here, cross-sectional view 500 is shown, including ETT 502 and lumen 504, the latter of which may be adapted for receiving a stylet imaging system 200 into connector mouth 216 (FIG. 2). In some examples, cross-sectional view 500 may also represent the proximal end of stylet 502, image processor 206, display 208, and any other proximally-coupled components are removed. Here, lumen 504 extends longitudinally within ETT 502 and region 506 disposed between the exterior surface of ETT 502 and lumen 504 may be comprised of rubber, plastic, PVC, or any other flexible material. An alternative cross-sectional view is shown in FIG. 5B.

FIG. 5B illustrates an alternative exemplary cross-sectional diagram of an airway management system. In this example, alternative cross-sectional view 510 is shown, including two lumens 504 within ETT 502. Region 508 may be implemented using rubber, plastic, PVC or any other flexible material. Either or both of lumens 504 may be used to receive a stylet imaging system (e.g., 200 or 211). Each of lumens 504 may also be used to deliver a gas (e.g., anesthesia), provide suction to remove secretions or other material from an airway, or receive a second stylet having another imaging device 204, light or coupled component. In other examples, more lumens may be provided. A cross-sectional view of the distal end of an airway management system is shown in FIG. 6.

FIG. 6 illustrates an exemplary transverse view of a distal end of an airway management system. Here, transverse view 600 includes ETT 602, imaging device 604 disposed at the tip of the distal end of stylet 606. Transverse view 600 illustrates the distal end of an airway management system (e.g., 211, 300). In other examples, different or additional devices may be used in place of imaging device 604 at the distal end of stylet 606. In some examples, imaging device 604 may be implemented as a camera, CCD, or other type of image capture device. Images captured by imaging device 604 may be converted to electrical signals and communicated using stylet 604 to other components (e.g., image processor 206 or 304, a transmitter, or local display).

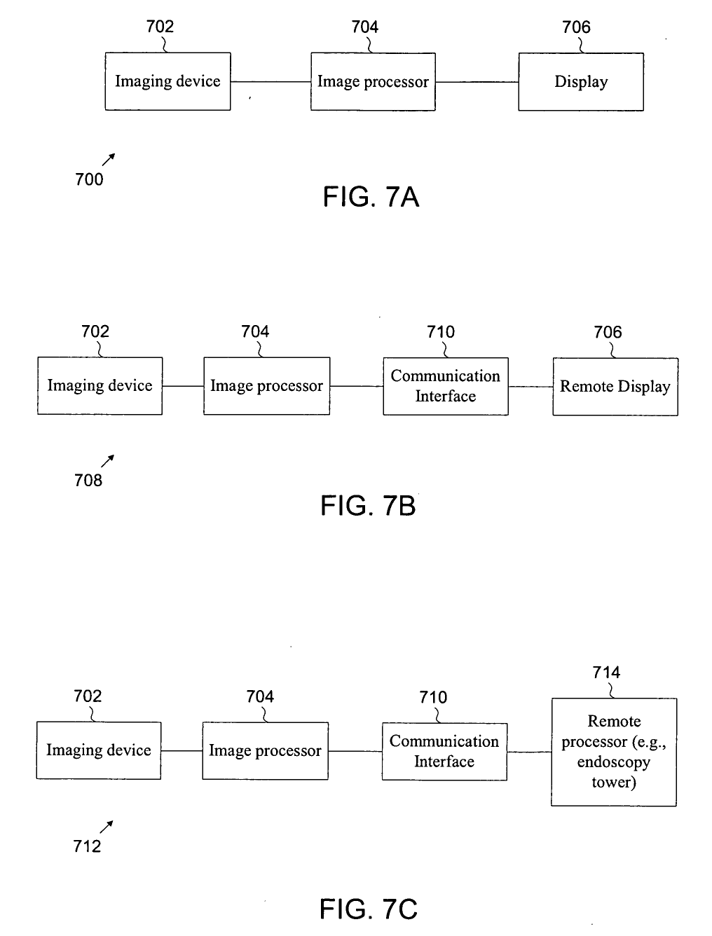

FIG. 7A is a block diagram illustrating an exemplary airway management system. Here, a block diagram is shown illustrating various airway management system 700 components, including imaging device 702, image processor 704, and display 706. In some examples, image processor 704 may also be referred to interchangeably as an image processing unit (IPU). Image processor 704 may include a memory (e.g., database, memory array, or other storage device) for storing data associated with electrical signals transmitted from an imaging device or other apparatus attached to the distal end of a stylet imaging system. In other examples, image processor 704 may also include software (i.e., computer programs) for executing a series of instructions or providing on-screen indications at a display attached to the proximal end of a stylet imaging system. In still other examples, an analog-to-digital converter may be used to convert signals from a data collection unit connected to the distal end of a stylet imaging system. As an example, a data collection unit may include a device that captures audio, video, and still images and sends associated signals to image processor 704 for resolution into images, video, or audio at display 706. In still other examples, image processor 704 may include a storage device, or memory for storing images (i.e., data) and other data from imaging device 702. In other examples, more or fewer components may be included, as illustrated in FIGS. 7B and 7C.

FIG. 7B is a block diagram illustrating an alternative exemplary airway management system. In this example, airway management system 708 also includes imaging device 702, image processor 704, communication interface 710, and remote display 706. Here, communication interface 710 provides capabilities to send and receive signals from image processor 704 to remote display 706. In this example, remote display 706 may be a large screen display, remotely located with an endoscopy tower (not shown), in the vicinity of an operating room, or another remote location. In another example, remote display 706 may also be existing monitors in a surgical operating room or facility, a room intended for observation of surgical operations, or any other location that may be in data communication with an airway management system. Specialized goggles or other optical devices may also be used as a remote display, receiving electrical signals directly from (via wireless RF signals) image processor 704 or communication interface 710 (e.g., wired or wireless). Here, remote display 706 enables viewers to observe positioning and placement of an ETT during intubation, perhaps observing or being able to provide input to the person (e.g., anesthesiologist) conducting the intubation. In other examples, two-way or networked communications may be provided that allow for remote or local viewing.

FIG. 7C is a block diagram illustrating yet another alternative exemplary airway management system. As an example, airway management system 712 may include imaging device 702, image processor 704, and communication interface 710 may be in data communication with remote processor 714. In some examples, remote processor 714 may be implemented using an endoscopy tower, which may be a collection of rack-mounted (or cart-mounted) systems. In some examples, an endoscopy tower may include systems or components for lighting, imaging, processing, monitoring, power supply, printing, or other endoscopic functions. A remote processor may also be a computer or server used to execute a series of instructions or processes for operating an airway management system, such as those examples described above. In the examples of FIGS. 7A-7C, electrical signals between imaging device 702 and image processor 704 are conducted using stylet imaging system 200 or the like, as described above. Signals conducted between image processor 704 (FIG. 7A) and display 706 or communication interface 710 (FIGS. 7B-7C) may also occur using stylet imaging system 200.

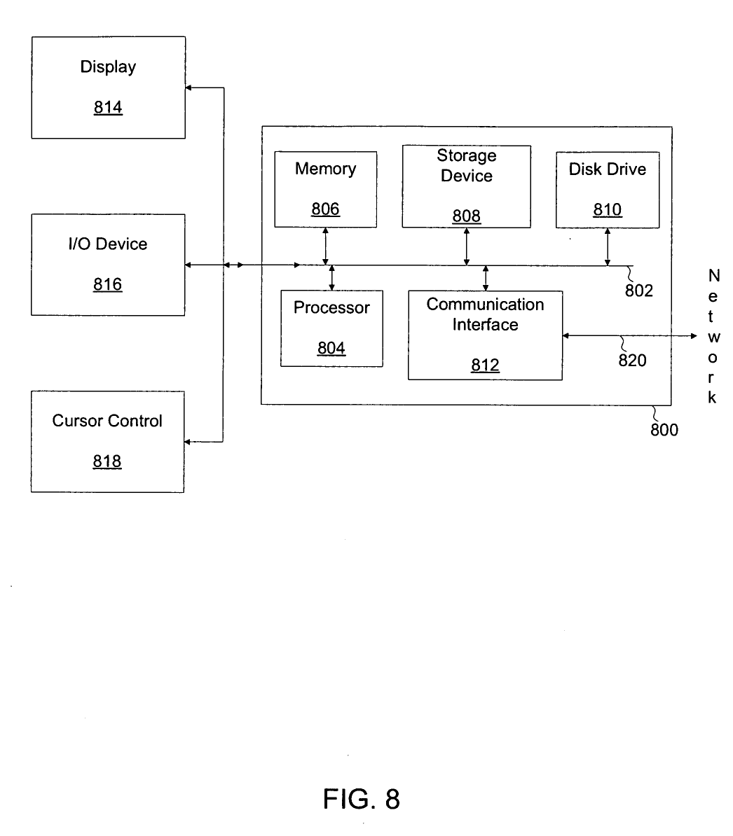

FIG. 8 is a block diagram illustrating an exemplary computer system suitable for implementing airway management. In some examples, computer system 800 may be used to implement the above-described techniques as processes or sets of instructions embedded in computer software or hardware. Here, computer system 800 includes a bus 802 or other communication mechanism for communicating information, which interconnects subsystems and devices, such as processor 804, system memory 806 (e.g., RAM), storage device 808 (e.g., ROM), disk drive 810 (e.g., magnetic or optical), communication interface 812 (e.g., modem or Ethernet card), display 814 (e.g., CRT or LCD), input device 816 (e.g., keyboard), and cursor control 818 (e.g., mouse or trackball).

According to one embodiment of the invention, computer system 800 performs specific operations by processor 804 executing one or more sequences of one or more instructions contained in system memory 806. Such instructions may be read into system memory 806 from another computer readable medium, such as static storage device 808 or disk drive 810. In alternative embodiments, hard-wired circuitry may be used in place of or in combination with software instructions to implement the invention.

The term “computer readable medium” refers to any medium that participates in providing instructions to processor 804 for execution. Such a medium may take many forms, including but not limited to, non-volatile media, volatile media, and transmission media. Non-volatile media includes, for example, optical or magnetic disks, such as disk drive 810. Volatile media includes dynamic memory, such as system memory 806. Transmission media includes coaxial cables, copper wire, and fiber optics, including wires that comprise bus 802. Transmission media can also take the form of acoustic or light waves, such as those generated during radio wave and infrared data communications.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, carrier wave, or any other medium from which a computer can read.

In an embodiment of the invention, execution of the sequences of instructions to practice the invention is performed by a single computer system 800. According to other embodiments of the invention, two or more computer systems 800 coupled by communication link 820 (e.g., LAN, PSTN, or wireless network) may perform the sequence of instructions to practice the invention in coordination with one another. Computer system 800 may transmit and receive messages, data, and instructions, including program, i.e., application code, through communication link 820 and communication interface 812. Received program code may be executed by processor 804 as it is received, and/or stored in disk drive 810, or other non-volatile storage for later execution.

Although the foregoing embodiments have been described in some detail for purposes of clarity of understanding, the invention is not limited to the details provided. There are many alternative ways of implementing the invention. The disclosed embodiments are illustrative and not restrictive.

What is claimed:

1. A system for managing an airway, comprising:
an imaging device configured to capture an image of the airway and convert the image into a signal;
a stylet configured to guide placement of an endotracheal tube in the airway and conduct the signal from the imaging device, the stylet being configured for introduction into a lumen of the endotracheal tube, the stylet and endotracheal tube being inserted into the airway; and
an image processor configured to receive and process the signal from the imaging device, the image being sent to a display for use in guiding placement of the endotracheal tube.

2. The system recited in claim 1, wherein the stylet is flexible.

3. The system recited in claim 1, wherein the stylet is malleable.

4. The system recited in claim 1, wherein the stylet is rigid.

5. The system recited in claim 1, wherein the stylet is deformable.

6. The system recited in claim 1, wherein the stylet comprises an electrically conductive, flexible rod.

7. The system recited in claim 1, wherein the stylet is disposable.

8. The system recited in claim 1, wherein the stylet includes a lumen having a wire configured to conduct the signal.

9. The system recited in claim 1, wherein the display is locally coupled to the image processor.

10. The system recited in claim 1, wherein the display is remote from the system.

11. The system recited in claim 1, wherein the signal is communicated to a receiver, the receiver communicating the signal to a remote processor configured to resolve the image.

12. The system recited in claim 1, wherein the signal is communicated to an endoscopy tower.

13. The system recited in claim 1, wherein the stylet further comprises a wireless transceiver for transmitting and receiving the signal between the system and a remote device.

14. The system recited in claim 1, wherein the imaging device is coupled to the distal end of the stylet.

15. The system recited in claim 1, wherein the image processor is coupled to the proximal end of the stylet.

16. The system recited in claim 1, wherein the system is configured to supply anesthesia to the airway.

17. The system recited in claim 1, further comprising a laryngotracheal anesthesia module configured to supply anesthesia to the airway through the lumen.

18. The system recited in claim 1, wherein the system is coupled to a suction source configured to remove secretions from the airway through the endotracheal tube.

19. The system recited in claim 1, further comprising a sensor configured to monitor a parameter associated with the airway.

20. The system recited in claim 19, wherein the parameter is a gas concentration in the airway.

21. The system recited in claim 19, wherein the parameter is a temperature.

22. The system recited in claim 1, wherein the system secures the airway using a balloon, the balloon when inflated creating a seal between the endotracheal tube and the airway.

23. A stylet imaging system, comprising:
an electrically conductive and malleable stylet configured for insertion into an endotracheal tube;
an imaging device coupled to the distal end of the stylet;
an image processor coupled to the proximal end of the stylet; and
a display configured to present an image of an airway as the stylet imaging system is introduced into the endotracheal tube and inserted into the airway.

24. The stylet imaging system recited in claim 23, wherein the electrically conductive and malleable stylet is disposable.

25. A method for airway management, comprising:
capturing an image of the airway;
converting the image into a signal;
conducting the signal using an electrically conductive and malleable stylet;
processing the signal to display the image of the airway; and
guiding the insertion of an endotracheal tube into the airway using an airway management system, the airway management system having an imaging device configured to capture the image and a display for viewing the image.

26. The method recited in claim 25, wherein the electrically conductive and malleable stylet is disposable.
